(19) United States
(12) Patent Application Publication
(10) Pub. No.: US 2022/0273849 A1
(43) Pub. Date: Sep. 1, 2022

(54) Human Placental Membrane Based Hydrogel Composition, Processes and Uses Thereof

(71) Applicant: UNIVERSIDADE DE AVEIRO, Aveiro (PT)

(72) Inventors: João Filipe COLARDELLE DA LUZ MANO, Aveiro (PT); Catarina DE ALMEIDA CUSTÓDIO, Aveiro (PT); Sara Catarina NUNES DA SILVA SANTOS, Fornos CPV (PT); Inês ARAÚJO DEUS, Aveiro (PT)

(21) Appl. No.: 17/631,106

(22) PCT Filed: Jul. 31, 2020
(86) PCT No.: PCT/IB2020/057283

§ 371 Date: Jan. 28, 2022

(30) Foreign Application Priority Data
Jul. 31, 2019 (PT) 115689

(51) Int. Cl. A61L 27/52; A61K 35/12; A61L 27/36

(52) U.S. Cl. CPC A61L 27/52; A61K 35/12; A61L 27/3633

(57) ABSTRACT

The present disclosure relates to a hydrogel composition comprising a protein extract obtained from a decellularized human placental membrane with a polymerizable or assemblable moiety; and at least a photoinitiator. The method to obtain such composition, as well as the uses thereof are also described.

### TECHNICAL FIELD

The present disclosure relates to the field of medicine, namely to the use of human placental membrane-derived extracellular matrix in regenerative medicine, tissue engineering, as pro-angiogenic implantable devices, replacement biomaterials, drug delivery systems, platforms for 3D cell culture and disease modelling and other biomedical and biological applications.

### BACKGROUND

Hydrogels are versatile biomaterials capable of better mimicking the extracellular matrix (ECM) of native tissues, thus supporting cell growth ex vivo and in vitro. Therefore, they have been widely applied for biomedical, biotechnological and pharmaceutical purposes, as 2D and 3D cell culture platforms, injectable and delivery matrices, transplantable scaffolds, medical devices, printable scaffolds, among others.

Recently, ECM-derived hydrogels emerged to better mimic the extracellular microenvironment, in an attempt to replicate the composition of the original tissue. These novel biomaterials are superior when compared to commonly used ones, namely those composed of single ECM components. However, such materials are mostly animal-derived which brings concerns regarding their biocompatibility, immunogenicity, risk of disease transmission and also concerns related to ethical approval.

Human placental membrane (hPM) is responsible for protecting the fetus and allow the exchange of nutrients and metabolic products during pregnancy. It has been widely recognized as a promising biomaterial due to its rich content in collagens and other structural proteins, as well as in bioactive factors which confer it anti-inflammatory, anti-bacterial, non-immunogenic, anti-scarring, anti-adhesive and pro-epithelization properties.

hPM started to be applied in clinical fields as surgical dressings, protective barriers and grafts for organ reconstruction or replacement. More particularly, hPM was applied in dermatological and ophthalmological fields to repair burns and wounds, and less extensively to the surgical reconstruction of the vagina and prevention of postoperative adhesion. Currently, hPM has been explored as an acellular scaffold for tissue engineering and regenerative medicine. It was already proved the efficiency in the regeneration of several tissues (e.g. cartilage), either alone or combined with cells.

For many applications hPM possesses poor mechanical properties and unsuitable biodegradation rates. Some strategies have been applied in order to improve its stability, including crosslinking of decellularized hPM (dhPM) [1], multilayered constructs of hPM laminates [2], composite scaffolds of dhPM and electrospun fibers [3], composite scaffolds of solubilized hPM and polymeric hydrogels [4] and hydrogels produced from hPM-derived ECM [5]. However, there is no report of a hydrogel completely derived from hPM with improved stability and tunable mechanical properties that can be used for medical (e.g. injectable matrices) and research (e.g. cell culturing) purposes.

Therefore, there remains a need for a biomaterial that: (1) comprises the structural and mechanical properties of hydrogels (e.g. high water content, adequate porosity and mass transport properties, suitable elasticity); (2) comprises the composition and structure of native ECM, thus representing a more appropriate microenvironment for cell growth ex vivo and in vitro; (3) comprises the advantages of natural polymeric materials, including bioactivity and biolcyto-compatibility, without comprise their main disadvantages, namely poor stability and mechanical properties.

These facts are disclosed in order to illustrate the technical problem addressed by the present disclosure.

### GENERAL DESCRIPTION

The present disclosure relates to a novel process of making protein extracts from hPM to obtain a hydrogel composition, process and uses thereof. The present disclosure relates to the modification of dhPM to produce hydrogels with controllable mechanical properties and stability with potential application in regenerative medicine, tissue engineering, as pro-angiogenic implantable devices, replacement biomaterials, drug delivery systems, platforms for 3D cell culture and disease modelling and other biomedical and biological applications. The major advantage of such materials is the opportunity to create hydrogels without complicated synthesis for bioconjugation. Moreover, these hydrogels have less risk of cross reactivity, immune reaction or disease transmission due to the properties of dhPM (e.g. non-immunogenicity). The process to obtain hPM-derived hydrogels is summarized in three major steps: (1) hPM isolation, decellularization and solubilization, (2) modification of the solubilized hPM and (3) formation of hPM-based hydrogels by photopolymerization.

The present disclosure relates to bioactive hydrogels derived from hPM. The disclosure further relates to hydrogel-based biomaterials applicable to biological, biomedical, biotechnological and pharmaceutical fields. More particularly the disclosure relates to medical/implantable devices, cell culture platforms, delivery matrices, injectable systems and 3D printable scaffolds.

The present disclosure relates to hydrogel-based materials composed of a polymeric network containing ECM components, namely hPM-derived extracellular matrix (hPM-ECM), which is bioactive due to its increased content in some components like collagen, laminin or fibronectin.

More particularly, the present disclosure relates to the modification of hPM-ECM derived materials with a chemical agent that allows for further chemical or physical crosslinking to create a hydrogel for use in biomedical, biotechnological and pharmaceutical applications. The present disclosure relates to a novel process for making a crosslinked hPM-ECM derived hydrogel, which shows increased stability compared to ECM-based, collagen-based and basement membrane-based hydrogels and sealants of the art. The hydrogels produced by the methods of the present disclosure provide the necessary structural and biochemical support for cell growth and are preferably 3D, and particularly suitable for cell culture and drug/cell delivery.

The present disclosure relates to hPM-based hydrogels to be applied in regenerative medicine and tissue engineering. More particularly the present disclosure relates to medical/implantable devices, cell culture platforms, delivery matrices, injectable systems and 3D printable scaffolds.

The present disclosure relates to a hydrogel composed of a polymeric matrix containing ECM proteins. More particularly, the present disclosure relates to the modification of hPM-ECM derived proteins with a chemical agent that allows for further chemical or physical crosslinking to create hydrogels. The hydrogels produced by the methods of the present disclosure provide structural and biochemical support for cell growth and are preferably 3D, and particularly suitable for cell culture and drug/cell delivery.

The present disclosure provides a composition comprising hPM-derived components functionalized by at least one polymerizable moiety and methods of use thereof. In one embodiment the composition comprises ECM proteins, cytokines, growth factors and other components that improve cell adhesion and growth. The present disclosure further improves tissue regeneration and restoration, comprising bio- and cytocompatibility.

Surprisingly the composition of the present disclosure has superior mechanical properties.

An aspect of the present disclosure is related to a hydrogel composition comprising:
a protein extract obtained from a dhPM with a polymerizable or assemblable moiety; and at least a photoinitiator; wherein the polymerizable or assemblable moiety is selected from a list consisting of: a methacrylate, acrylate, ethacrylate, acryloyl, thiol, acrylamide, aldehyde, azide, cyclic oligosaccharides, phenol, phenol derivatives, or combinations thereof; and
wherein the polymerizable or assemblable moiety is bound to the protein extract obtained from a decellularized human placental membrane.

In an embodiment, the ratio of protein extract: polymerizable or assemblable moiety is from 10:1×10⁻⁵ (v/v) to 10:1×10⁻¹, particularly 10:1×10⁻³ to 10:1×10⁻² (v/v).

In an embodiment, the concentration of protein extract in the hydrogel composition varies from 1-15% w/V, preferably from 1%-5% w/V; more preferably 1-2.5% w/V.

In an embodiment, the protein extract comprises at least two of the following proteins keratin, collagen, desmoplakin, dermcidin and peroxiredoxin, or mixtures thereof. In a further embodiment, the protein extract comprises at least three of the following proteins keratin, collagen, desmoplakin, dermcidin and peroxiredoxin, or mixtures thereof. In a yet further embodiment, wherein the protein extract comprises at least four of the following proteins keratin, collagen, desmoplakin, dermcidin and peroxiredoxin, or mixtures thereof.

In an embodiment, the polymerizable or assemblable moiety is methacrylate.

In an embodiment, the polymerizable or assemblable moiety is a thiol, a methacrylate, or mixtures thereof.

In an embodiment, the polymerizable or assemblable moiety is a phenol or phenol derivative.

In an embodiment, the dhPM is selected from: amnion membrane, chorion membrane or combinations thereof.

In an embodiment, the composition comprises at least two different polymerizable or assemblable moieties.

In an embodiment, the hydrogel polymerization occurs by crosslinking, in particular crosslinking performed via chemical crosslinking, non-covalent bonds, including guest-host complexes or metallic coordination, or crosslinked enzymatically via transglutaminase, or combinations thereof.

In an embodiment, the photoinitiator is selected from a list comprising: 2-Hydroxy-4′-(2-hydroxyethoxy)-2-methylpropiophenone, acetophenone, benzil, benzophenone, 1-hydroxycyclohexyl phenyl ketone, among others.

In an embodiment, the protein extract is chemically modified with biodegradable linkages, in particular ester linkages, amide linkages, azide-alkyne cycloaddition linkages, acrylate-thiol linkages, urethane linkages, and/or methacrylate-thiol linkages and combinations thereof.

In an embodiment, the composition may further comprise inorganic materials selected from: calcium phosphate, magnetic particles, metallic nanoparticles, bioglass particles, fibers or combinations thereof.

In an embodiment, the composition may further comprise chitosan, alginate, laminarin, hyaluronic acid, polyethylene glycol, or combinations thereof.

An aspect of the present disclosure comprises a hydrogel precursor for obtaining the hydrogel composition described in any of the previous claims comprising: a protein extract obtained from a decellularized human placental membrane with a polymerizable or assemblable moiety; and optionally at least a photoinitiator, wherein the polymerizable or assembly moiety is selected from a list consisting of: a methacrylate, acrylate, ethacrylate, acryloyl, thiol, acrylamide, aldehyde, azide, cyclic oligosaccharides, phenol, phenol derivatives, or combinations thereof; and wherein the polymerizable or assemblable moiety is bound to the protein extract obtained from a decellularized human placental membrane.

In an embodiment, the protein extract is an autologous protein from the patient to treat. In the present disclosure, “autologous protein” is described as a protein obtained from the same individual that is intended to receive such proteins.

Another aspect of the present disclosure relates to a microparticle, powder, capsule, fiber, membrane, disc, geometrically controlled microgel, sponge, lyophilizate, organ-on-a-chip, printable construct, an ointment, a mesh, a foam, a scaffold or a delivery matrix comprising the hydrogel composition or the hydrogel precursor disclosed in the present subject matter.

Another aspect of the present disclosure relates to device or system, lab-on-a-chip, microscopy or microarray system comprising the hydrogel composition or hydrogel precursor disclosed in the present subject matter, particularly microwell plates, microfluidic or sampling and microparticles.

Another aspect of the present disclosure relates to substrate comprising the hydrogel composition or hydrogel precursor disclosed in the present subject matter.

An aspect of the present disclosure relates to cell and tissue culture dishes comprising the hydrogel composition or hydrogel precursor disclosed in the present subject matter.

Another aspect of the present disclosure relates to a delivery matrix comprising the hydrogel composition or hydrogel precursor disclosed in the present subject matter wherein the matrix is loaded with biological active agents or therapeutic agents, including cells, stem cells, proteins, vaccines, biomolecules, diagnostic markers, probes or combinations thereof.

Another aspect is the use in medicine or veterinary medicine of the hydrogel composition or hydrogel precursor described in the present subject matter. Preferably, in pharmaceutical studies, biotechnological processes, or ex vivo and in vitro studies. More in particular, the use of the hydrogel composition or hydrogel precursor in regenerative medicine, tissue engineering, as pro-angiogenic implantable devices, replacement biomaterials, drug delivery, platforms for 3D cell culture and disease modelling and other biomedical and biological applications.

Use of the hydrogel composition or hydrogel precursor for cell culture, encapsulation of living cells, drug delivery, cell delivery, cell regeneration, organ development and tissue growth.

Another aspect of the present disclosure is related to a method for obtaining a protein extract of dhPM as disclosed in the present subject matter comprising the following steps:
washing an isolated hPM;
decellularizing the washed membrane;
solubilizing the dhPM;
freezing, lyophilizing and/or grinding the solubilized membrane;
bounding to the protein extract at least one polymerizable or assemblable
moiety to obtain a reactively enhanced extract.

In an embodiment, the method further comprises the step of storing the functionalized protein extract at between around −80° C. and 4° C.

In an embodiment, the method further comprises the following steps:
conjugation with a photoinitiator, in particular a free-radical photoinitiator, selected from a list comprising: 2-Hydroxy-4′-(2-hydroxyethoxy)-2-methylpropiophenone, acetophenone, benzil, benzophenone, 1-hydroxycyclohexyl phenyl ketone, among others;
chemically modifying the protein extract derived from the placental membrane with biodegradable linkages, which may include, for example, ester linkages, amide linkages, azide-alkyne cycloaddition linkages, acrylate-thiol linkages, urethane linkages, and/or methacrylate-thiol linkages and combinations thereof.

In an embodiment, the method comprises the step of terminally sterilizing the obtained protein extract derived from the placental membrane by means of filtration or chemical reaction, preferably with peracetic acid, ethylene oxide or supercritical CO₂; irradiation, preferably UV light, gamma radiation.

In an embodiment, the decellularization step is performed by means of detergent, enzymatical methods, chemical methods, physical methods, or combinations thereof, in order to become free of cells, enhancing bio- and cytocompatibility.

In an embodiment, the solubilization step is performed by enzymatic digestion, particularly with pepsin at a low pH, allowing the maintenance of the native bioactivity.

In an embodiment, the grinding step is reducing to powder.

An aspect of the present disclosure relates to a method for preparing the hydrogel composition as described in any of the previous claims, the method comprising: obtaining a functionalized protein extract of decellularized placental membrane; mixing said functionalized protein extract of decellularized placental membrane with a photoinitiator; and irradiating the mixture comprising the photoinitiator and the protein extract with light to promote the crosslinking of the hydrogel, preferably with UV light, for 0.5 to 5 minutes, preferably 1 minute.

In an embodiment, the crosslinking is performed via chemical crosslinking, non-covalent bonds, including guest-host complexes or metallic coordination, or crosslinked enzymatically via transglutaminase, or combinations thereof.

According to the present disclosure, dPM may be modified to enhance its chemical reactivity. The polymerizable moiety of the present disclosure may be selected, for example, from methacrylates, ethacrylates, thiols, acrylamides, aldehydes, azides, amine reactive groups or cyclic oligosaccharides and combinations thereof. In one embodiment the polymerization of the present composition comprising the modified dPM precursors occurs via chemical crosslinking, enzymatic crosslinking, metal coordination or other non-covalent assembly, or guest-host complexation, and combinations thereof, using appropriate crosslinking agents.

The mechanical properties of dPM-derived hydrogels of the present disclosure are increased as compared to similar non-modified compositions, and the properties may be easily tuned to fit the intended purpose. Moreover, the hydrogel according to the present disclosure further possesses improved stability towards enzymatic and proteolytic degradation when compared to similar non-modified compositions. In one embodiment, the present disclosure comprising tunable mechanical properties may be used to control biological responses by modifying the composition, crosslinking methodology and crosslinking density.

In a particular aspect, the present disclosure relates to a hydrogel of chemically or physically crosslinked dhPM-derived components network with said functional groups, dhPM-based hydrogels formed via non-covalent bonds, including guest-host complexes or metallic coordination, dhPM-based hydrogels crosslinked enzymatically using appropriate crosslinking agents.

In another aspect the present disclosure relates to a bioactive hydrogel for culture and encapsulation of living cells.

In a further aspect the present disclosure relates to 3D printable hydrogels and injectable systems comprising cells and the hydrogel described in the present disclosure.

In a further aspect the present disclosure relates to the use of the hydrogel according to the present disclosure in lab-on-a-chip systems, microscopy and microarray substrates, cell and tissue culture dishes, microwell plates, microfluidic or sampling and microparticles.

In one embodiment, the composition is a hydrogel. In one embodiment the composition is a powder. In one embodiment the composition is a sponge. In one embodiment the composition is a lyophilizate. In one embodiment the composition is a scaffold.

The present disclosure may be applied as a biomaterial, in particular it may be used as a biomaterial in medicine, pharmaceutical studies, biotechnological processes, or ex vivo and in vitro studies. In some instances, the referred composition is configured as a cell culture platform and cell encapsulation matrix for research or commercial purposes. In some instances, the referred composition is configured as a delivery matrix which can be loaded with biological active agents or therapeutic agents, including but not limited to cells, stem cells, proteins, vaccines, biomolecules, diagnostic markers and probes and combinations thereof. In some instances, the referred composition is configured as an injectable system for tissue engineering and regenerative medicine. In some instances, the referred composition is configured as a printable scaffold. In some instances, the referred composition is configured as an implantable construct. In some instances, the referred composition is configured as a lab-on-a-chip or microfluidic system. In some instances, the referred composition is configured as nanoparticles, microparticles, microgels, capsules, fibers, membranes, discs, patches, among others.

In one embodiment the composition comprising hPM components is decellularized. The decellularization is achieved by means of detergent, enzymatical, chemical or physical methods and combinations thereof, this way becoming free of cells enhancing bio- and cytocompatibility.

In one embodiment the composition comprising dhPM components is solubilized. The solubilization is achieved by means of enzymatic digestion with, for example, pepsin at a low pH, allowing the maintenance of the native bioactivity. In some instances, the solubilized composition comprising dhPM components may be further frozen, lyophilized and powdered.

In one embodiment the composition comprising dhPM components is chemically modified. In some instances, the chemical modification of the present composition comprises a photoreactive moiety (e.g. acrylate, methacrylate or acryloyl groups) and polymerizes upon ultraviolet (UV) light exposure. In some instances, the composition comprising dhPM components conjugated with photoreactive moieties is further combined with a photoinitiator, in particular a free-radical photoinitiator, selected from the following list: 2-Hydroxy-4′-(2-hydroxyethoxy)-2-methylpropiophenone, acetophenone, benzil, benzophenone, 1-Hydroxycyclohexyl phenyl ketone, among others. In some instances, the chemical modification of the present composition comprises biodegradable linkages, which may include, for example, ester linkages, amide linkages, azide-alkyne cycloaddition linkages, acrylate-thiol linkages, urethane linkages, and/or methacrylate-thiol linkages and combinations thereof.

In one embodiment the composition comprising hPM components is terminally sterilized. In some instances, the present composition is in the soluble format and is terminal sterilized by means of filtration or chemical reaction. In some instances, the present composition is in the solid format and is terminal sterilized by means of irradiation (e.g. UV light, gamma radiation) or chemical reaction (e.g. ethylene oxide, supercritical CO₂).

In one embodiment the composition comprises the modified dhPM precursors at a range of concentrations from 1% to 15% (w/V), preferably 1%, 2.5% or 5% (w/V).

In one embodiment the composition comprises the modified dhPM precursors combined with another natural or synthetic based polymer, such as chitosan, alginate, laminarin, hyaluronic acid or polyethylene glycol (PEG). In one embodiment the composition comprises the modified dhPM precursors combined with inorganic materials such as calcium phosphate, metallic nanoparticles, magnetic particles or bioglass particles or fibers.

### BRIEF DESCRIPTION OF THE DRAWINGS

The following figures provide preferred embodiments for illustrating the description and should not be seen as limiting the scope of disclosure.

FIG. 1—dhPM-derived hydrogels fabrication process. After hPM isolation, decellularization, digestion and modification, dhPM hydrogels are formed upon crosslinking of the polymerizable or assemblable moieties.

FIG. 2—A) a. Fresh amniotic membrane (AM) after isolation and cleaning; b. Decellularized AM (dAM) after treatment with SDS, Triton X 100 and nucleases; c. DAPI staining showing the presence of cell nucleus in the native AM; d. DAPI staining showing the lack of cell nucleus in the AM after decellularization. B) Histomorphological analysis of fresh and decellularized AM using H&E, Masson's trichome and Alcian blue stainings. Symbols: (↓) Basement membrane, (*) Stromal layer, (×) Collagen (●) GAGs. Scale bar=100 μm. C) DNA content decrease from fresh AM to dAM. D) Quantification of collagen and GAGs content before and after decellularization.

FIG. 3—A) Schematic illustration of AM reaction with methacrylic anhydride and AM methacrylate (AMMA) hydrogel preparation using irgacure 2959 as a photoinitiator and exposure to UV light. B) UV-Vis absorption spectra of dAM, AMMA100 and AMMA300 that resulted from TNBSA assay. C) Representative images of photopolymerized AMMA hydrogels prepared from low modification degree (AMMA100) and high modification degree (AMMA300) samples at 2.5% and 5% (w/v).

FIG. 4—Rheometer analysis of AMMA hydrogels at 1%, 2.5% and 5% (w/v). Representative curves of storage modulus (G′) increase over time for A) AMMA100 and B) AMMA300 samples; C) Graphical representation of G′ and loss modulus (G″) mean values obtained for each sample.

FIG. 5—Mechanical properties of AMMA100 and AMMA300 hydrogels at 2.5% and 5% (w/v). A) Representative compressive stress strain curves; B) Young's modulus calculated from compressive stress-strain curves; Mean values of C) ultimate strain D) ultimate stress and E) water content obtained for each sample. Statistical analysis through one-way ANOVA showed significant differences (*p<0.05) between the analysed groups.

FIG. 6—Representative fluorescence images of top seeding experiments with ASCs and HUVECs. Cell viability was assessed by live/dead assay at 1 and 7 days of culture. Cell morphology was assessed with DAPI/Phalloidin staining at 7 days of culture. Scale bar=100 μm.

FIG. 7—Representative fluorescence images of encapsulation experiments with ASCs and HUVECs. Cell viability was assessed by live/dead assay at 1 and 7 days or 3 and 7 days of culture for ASCs and HUVECs, respectively. Cell morphology was assessed with DAPI/Phalloidin staining at 7 days of culture. Scale bar=100 μm.

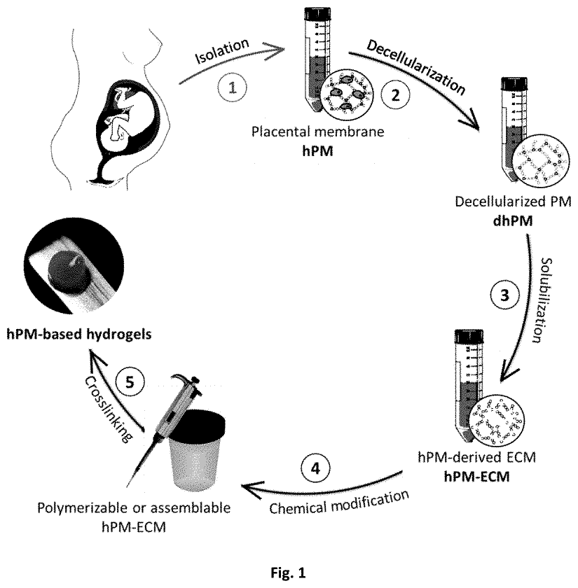

### DETAILED DESCRIPTION

The present disclosure provides a composition comprising hPM derived components, methods of processing and uses thereof. The present disclosure further provides methods to functionalize the composition described herein and uses thereof.

In one embodiment the hPM has to be isolated from the whole placenta. The isolated hPM has to be further processed to remove any blood clots and vessels. The cleaning of the referred sample may comprise the use of, for example, distilled water or saline solution. The hPM is chopped into smaller pieces to facilitate the following process.

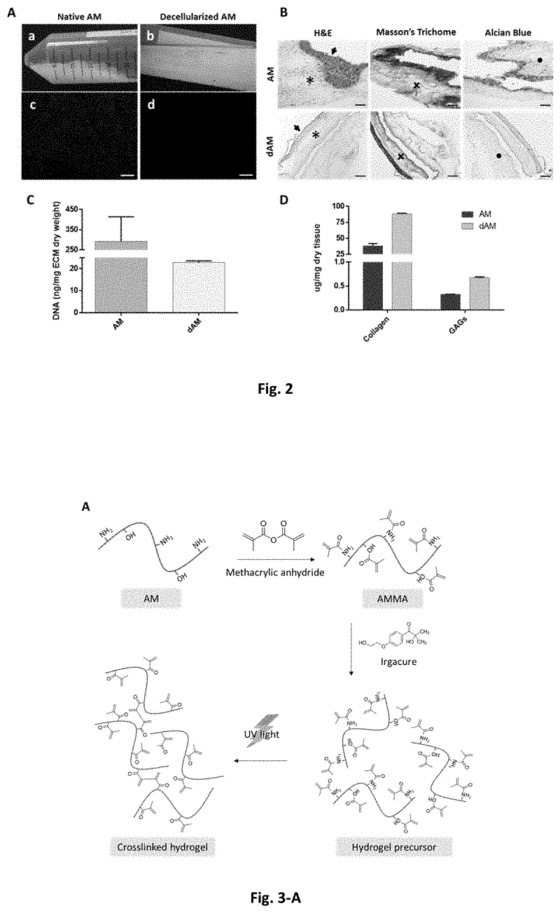

In one embodiment the hPM membrane is decellularized. The method of decellularization comprises the following steps: treat the hPM sample with an ionic detergent (e.g. sodium dodecyl sulfate) and a non-ionic detergent (e.g. Triton X-100) to promote the disruption of cell membranes; incubate the hPM sample with nucleases solution, comprising a reaction buffer, RNase and DNase, to remove nuclear debris. The composition resulting from the process described herein comprises hPM derived components, namely ECM components, free of cells and cellular debris. The efficiency of the described process may be accessed by quantification of deoxyribonucleic acid (DNA) per mg of ECM (dry weight), quantification of base pair DNA fragment length or visualization of the nuclear material present in the tissue by means of histological staining, such as haematoxylin and eosin (H&E) staining or 4′,6-dimidino-2-phenylindole (DAPI) staining. (FIG. 2)

In one non-limiting embodiment, the hPM used was the amniotic membrane (AM).

Efficient decellularization was confirmed by the lack of cell nucleus upon H&E and DAPI staining and a significant decrease in DNA content (FIGS. 2A, B and C). The preservation of key structural elements was further confirmed by histomorphologic analysis using Masson's trichome and Alcian blue stainings, quantification of collagen and glycosaminoglycans (GAGs) content (FIG. 2D).

In an embodiment, AM, dAM, and AMMA300 were characterized by mass spectrometry analysis. The main components found are listed in Table 1 according to their relative abundance.

TABLE 1
Main components of AM, dAM, and AMMA300, listed
according to their relative abundance, as
analysed by mass spectrometry analysis.

| | AM | dAM | AMMA300 |
|---|---|---|---|
| N | Protein | Protein | Protein |
| 1 | Keratin, type II | Collagen alpha-1(I) | Keratin, type II |
| | cytoskeletal 2 | chain | cytoskeletal 2 |
| | epidermal | | epidermal |
| 2 | Keratin, type II | Collagen alpha-1(III) | Keratin, type I |
| | cytoskeletal 1 | chain | cytoskeletal 10 |
| 3 | Keratin, type II | Collagen alpha-2(I) | Keratin, type II |
| | cytoskeletal 5 | chain | cytoskeletal 1 |
| 4 | Keratin, type I | Keratin, type II | Collagen alpha-2(I) |
| | cytoskeletal 10 | cytoskeletal 1 | chain |
| 5 | Keratin, type I | Keratin, type I | Keratin, type I |
| | cytoskeletal 9 | cytoskeletal 10 | cytoskeletal 9 |
| 6 | Desmoplakin | Keratin, type II | Keratin, type I |
| | | cytoskeletal 2 | cytoskeletal 16 |
| | | epidermal | |
| 7 | Serum albumin | Keratin, type I | Keratin, type II |
| | | cytoskeletal 9 | cytoskeletal 5 |
| 8 | Keratin, type I | Keratin, type I | Collagen alpha-1(I) |
| | cytoskeletal 16 | cytoskeletal 16 | chain |
| 9 | Keratin, type I | Keratin, type II | Keratin, type II |
| | cytoskeletal 17 | cytoskeletal 5 | cytoskeletal 6A |
| 10 | Keratin, type II | Keratin, type II | Keratin, type I |
| | cytoskeletal 6C | cytoskeletal 6A | cytoskeletal 14 |
| 11 | Keratin, type I | Keratin, type I | Desmoplakin |
| | cytoskeletal 19 | cytoskeletal 14 | |
| 12 | Annexin A2 | Dermcidin | Dermcidin |
| 13 | Actin, cytoplasmic 2 | Collagen alpha-1(II) | Collagen alpha-1(XI) |
| | | chain | chain |
| 14 | Prelamin-A/C | Taperin | Taperin |
| 15 | Endoplasmic | Hornerin | Peroxiredoxin-1 |
| | reticulum | | |
| | chaperone BiP | | |
| 16 | Neuroblast | Leucine zipper | Keratin, type II |
| | differentiation- | putative tumor | cytoskeletal 6B |
| | associated protein | suppressor 2 | |
| | AHNAK | | |

In one embodiment the dhPM is solubilized. In some instance, dhPM sample may be solubilized. In some instance, dhPM sample may be frozen, lyophilized and grinded previously to the solubilization. The method of solubilization comprises the incubation of dhPM sample with an enzymatic solution. In some instances, the enzymatic solution can comprise pepsin at a low pH, obtained from the addition of, for example, hydrochloric acid (HCl). In some instances, the solubilized sample is lyophilized or re-lyophilized.

In one embodiment the sample resulting from the method described herein is stored at low temperatures, preferably +4° C. or −80° C.

In one embodiment the composition of the present disclosure comprises a polymeric matrix containing amine groups. In some instances, methacrylic anhydride is added to the composition described herein to react with amine groups present in all proteins and add acrylate pendant groups. The reaction described herein occurs rapidly and with high yield. The reaction described herein is performed at a controlled temperature, in particular at 18-25° C. The composition resulting from the process described herein becomes a photopolymerizable biomaterial comprising photoreactive precursors.

In one embodiment the physicochemical and biological properties of the composition comprising the dhPM-derived components may be tailored, accordingly to the intended application, by variation of the ratio dhPM:methacrylic anhydride (which stands for methacrylation degree). In some instances, the physicochemical and biological properties referred herein may be further controlled with irradiation time and concentration of photoreactive precursors.

In one embodiment the degree of methacrylation may be accessed using the following method or methods: ¹H NMR, mass spectroscopy, 2,4,6-trinitrobenzene sulfonic acid colorimetric assay (TNBSA), fluoraldehyde assay, Habeeb method.

In one embodiment the composition of the present disclosure is a hydrogel or a hydrogel-based biomaterial. In some instances, the physicochemical and biological properties of the present hydrogel or hydrogel-based biomaterial may be tailored with the method described herein. In some instances, the controllable physicochemical and biological properties referred herein may be chosen from, for example, strength, stiffness, toughness, durability, degradability, mass transport and water uptake.

In one embodiment different ratios of dAM:methacrylic anhydride were tested, in particular the following ratios: 10:1×10⁻¹ (v/v), 10:1×10⁻³ (v/v) and 10:1×10⁻⁵ (v/v). Different concentrations of photoreactive precursors were further tested for each one of the ratios described, in particular the following concentrations: 1%, 2.5% and 5%. In one non-limiting embodiment the present compositions were further polymerized under UV light for 60 seconds to form hydrogels.

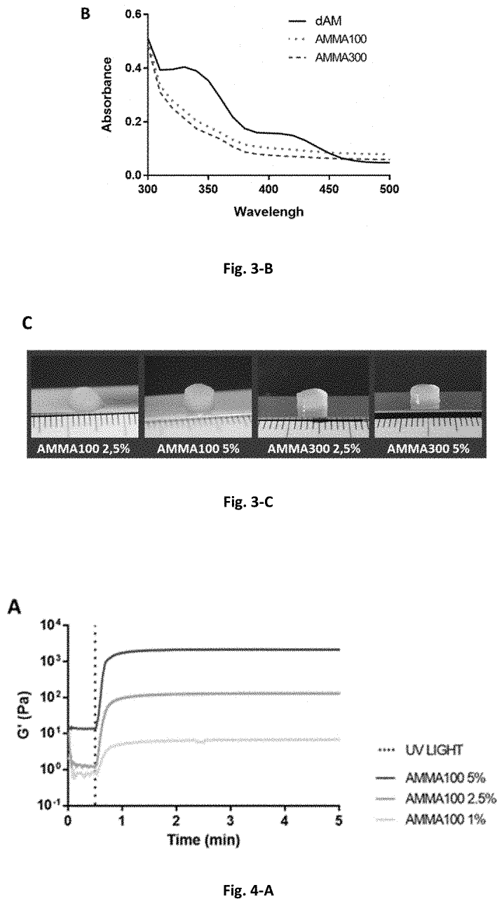

In one embodiment a photoinitiator (2-Hydroxy-4′-(2-hydroxyethoxy)-2-methylpropiophenone) is added to the photopolymerizable biomaterial to promote its polymerization when exposed to UV light at mild temperatures. (FIGS. 3A and C).

In one embodiment, the insertion of reactive groups in the dAM was verified by 2,4,6-trinitrobenzene sulfonic acid (TNBSA) colorimetric assay performed before and after modification. Chemical modification was confirmed by the decrease in the number of free amino groups after functionalization—samples AMMA100 and AMMA300 (FIG. 3B).

In one embodiment, the insertion of methacrylate groups was further confirmed by mass spectroscopy.

In one embodiment, rheological and mechanical characterization was performed on AM methacrylate samples with low (AMMA100) and high (AMMA300) methacrylation degree at concentrations of 1%, 2.5% and 5% (w/v), to assess the effects of functionalization and hydrogel precursor concentration on hydrogel mechanical properties.

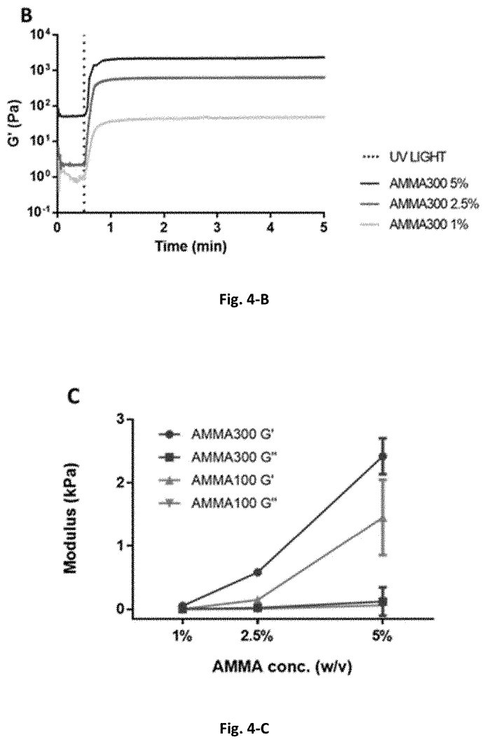

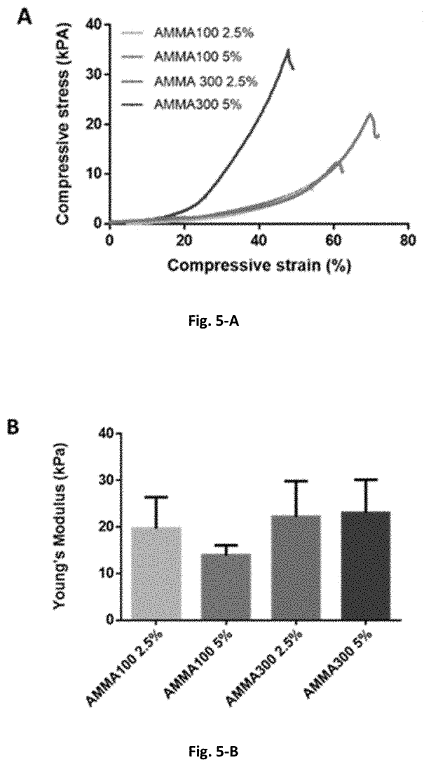

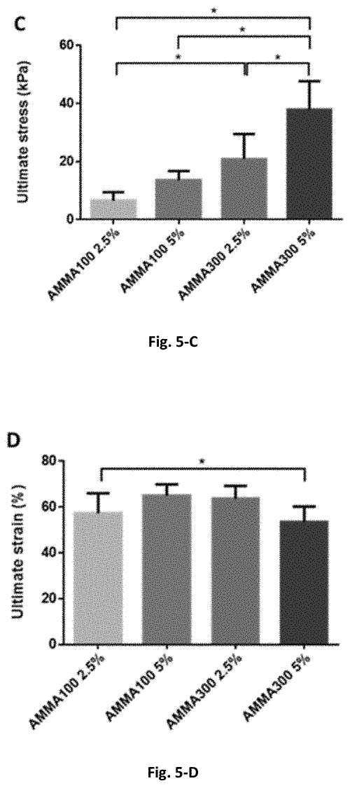

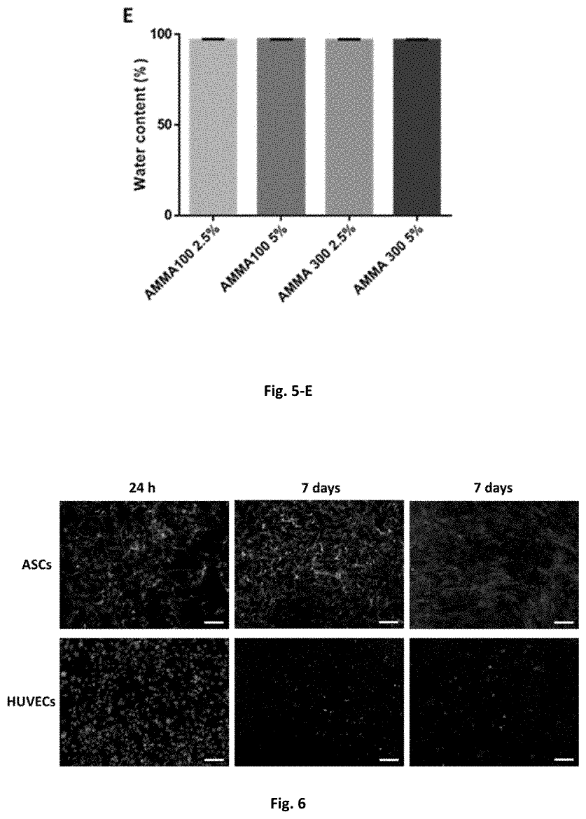

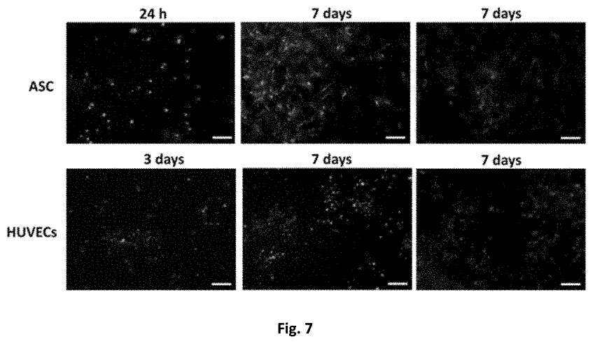

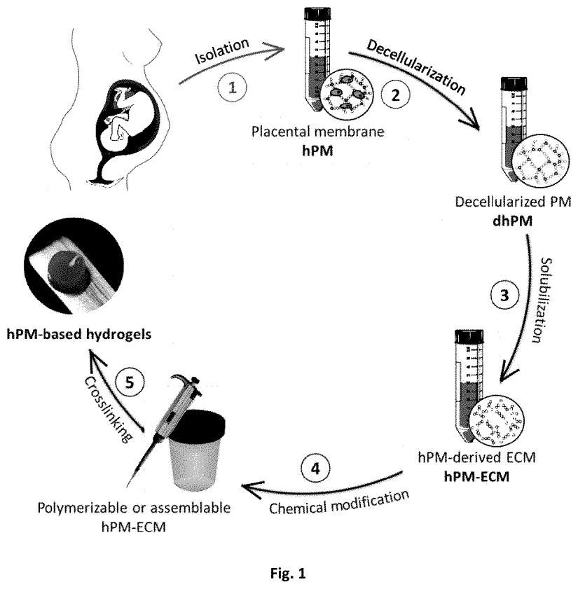

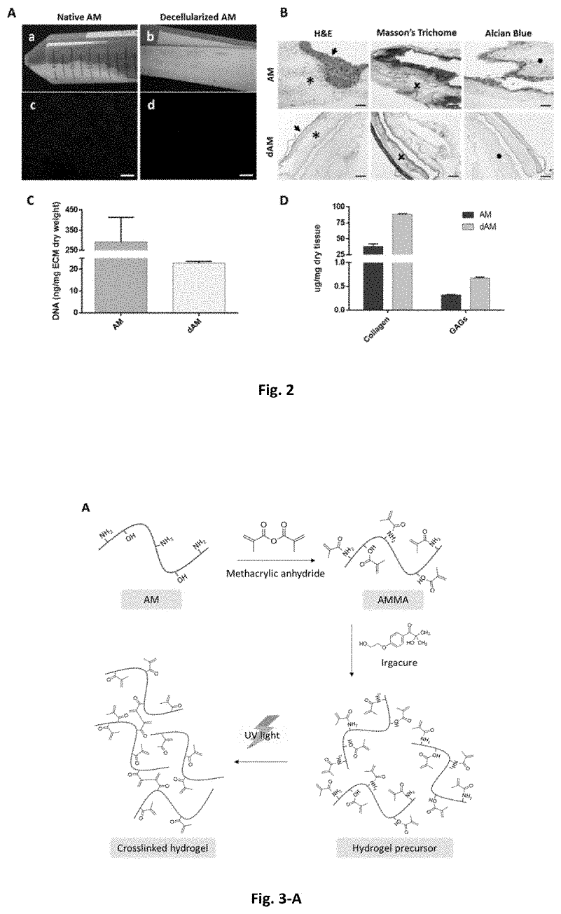

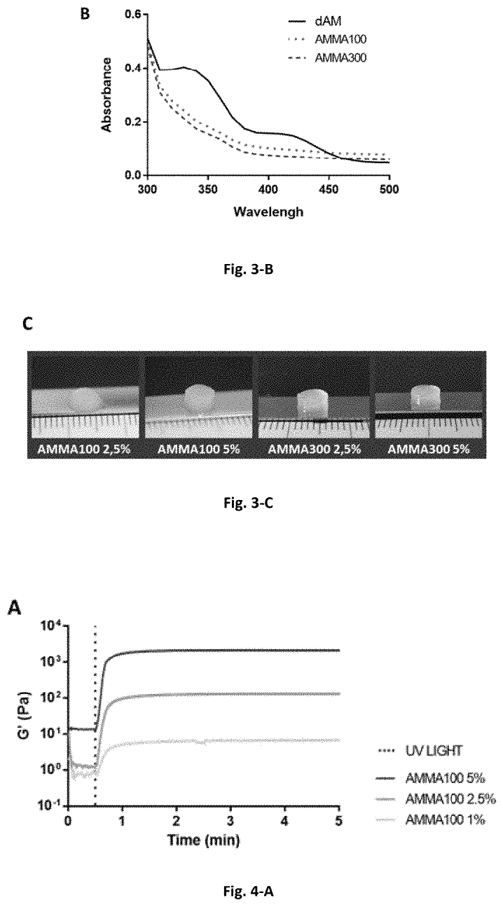

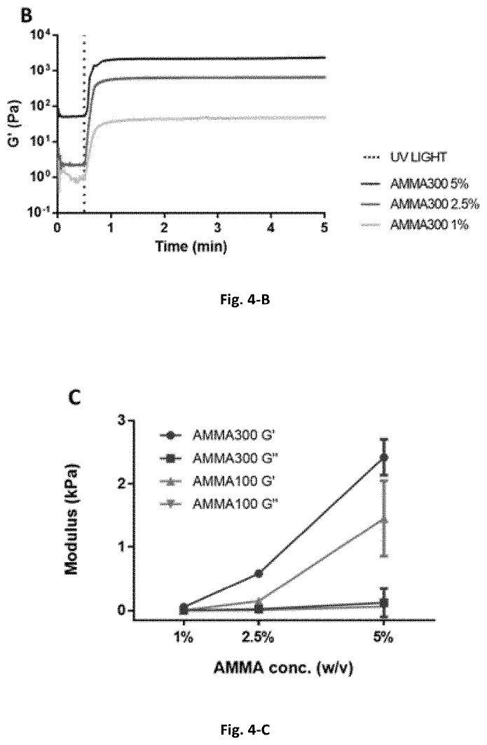

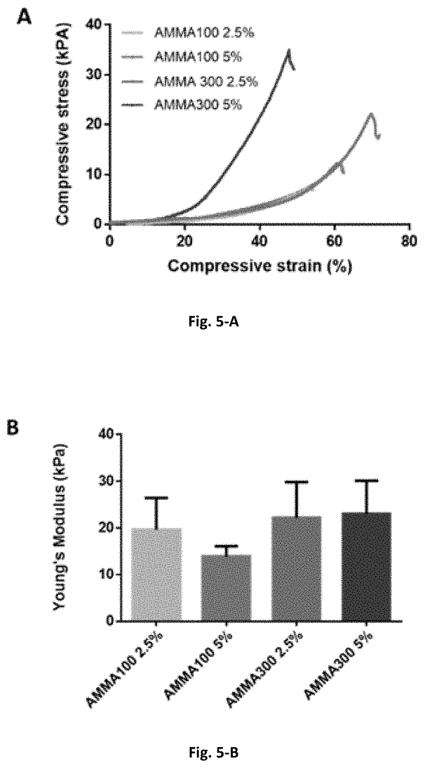

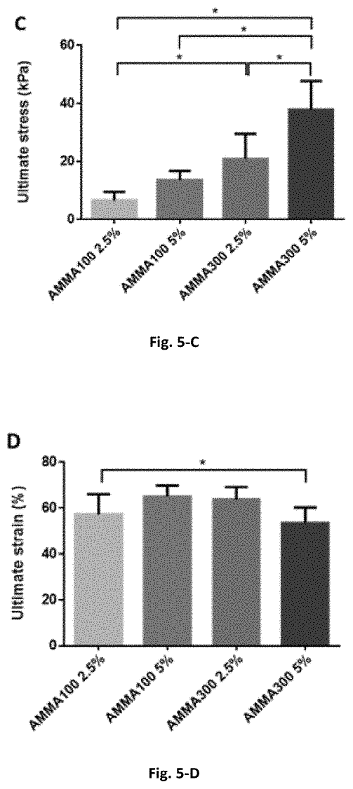

In general, increasing the hydrogel precursor concentration increased the stiffness of hydrogels formed (FIG. 4). Apparently, maintaining a constant hydrogel precursor concentration while increasing the degree of methacrylation did not significantly altered the mechanical properties of the hydrogels, although more robust hydrogels were obtained (FIGS. 4 and 5). Table 2 lists the phase angle variations as a function of crosslinking density and ECM concentration (w/v).

TABLE 2
Phase angle variations as a function of crosslinking
density and ECM concentration (w/v).

Phase Angle δ (°)

| Conc. (w/v) | AMMA100 | AMMA300 |
|---|---|---|
| 1% | 5.01 ± 1.9 | 2.7 ± 0.9 |
| 2.5% | 2.69 ± 0.4 | 2.0 ± 0.3 |
| 5% | 2.54 ± 0.5 | 2.0 ± 0.8 |

In an embodiment, the water content of hydrogels was also evaluated. Results shown that this parameter is not significantly different between all the studied conditions. In general, AMMA hydrogels have 90% of water content (FIG. 5E).

In one embodiment the cell culture performance and cytocompatibility of the hydrogels produced by the method described herein were assessed by in vitro culture of human adipose-derived stem cells (hASCs) and human umbilical vein endothelial cells (HUVECs).

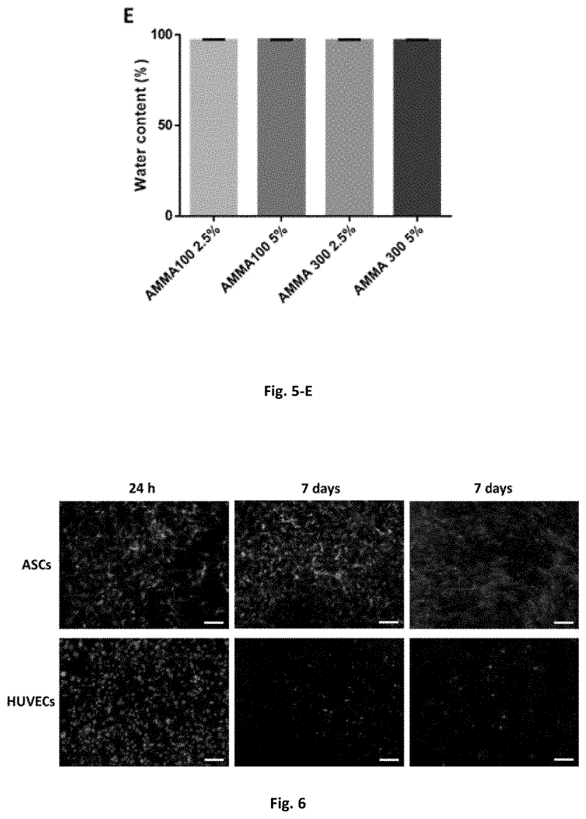

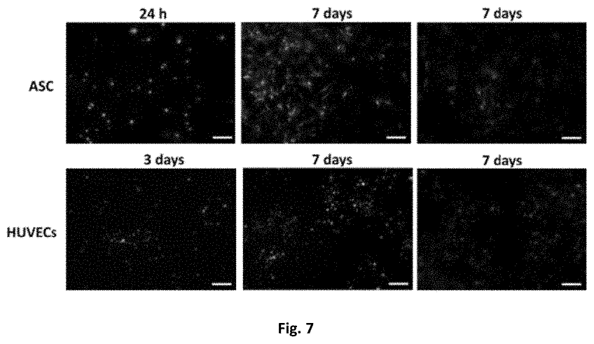

In one embodiment the viability, proliferation and morphology of cells seeded on top and encapsulated within the described hydrogels were assessed. In some instances, the hydrogel was pipetted into microwells and polymerized under UV light for 60 seconds. The referred cells were seeded on top or encapsulated inside of the hydrogels and incubated at 37° C. for different time periods, preferably periods of 24 hours, 3 days and 7 days, in cell culture medium. Cell viability was assessed at specific time-points using a live/dead staining. (FIGS. 6 and 7) Cell proliferation and morphology was assessed at specific time-points after hydrogel fixation, using DAPI/phalloidin staining (FIGS. 6 and 7). The present assays demonstrated the capacity of the hydrogel described herein to support cell adhesion and proliferation during several days in in vitro conditions.

The present disclosure relates to a scaffold produced from dhPM, which maintains most of the composition of the original tissue, thus providing an appropriate microenvironment for cells to adhere and growth. Differently from other ECM-derived substrates, the increased stability and tunable mechanical properties of the functionalized biomaterial described in the present disclosure, make it more suitable for multiple applications. Besides that, the present subject matter can be further combined with other materials and/or bioactive factors to enhance its biochemical and mechanical properties, which also enhances the range of possible applications, for example, as a delivery matrix or a graft material.

In one not-limiting embodiment, the present disclosure is preferably configured as a hydrogel that gels when exposed to UV light. The described hydrogel finds applicability in several purposes. In some instances, the present disclosure is used as a cell culture platform. For such purpose, the hydrogel can be first adsorbed into an appropriate platform (e.g. cell and tissue culture dishes, microwell plates), polymerized and then seeded with the desired cells or, alternatively, it can be first combined with the cells, then adsorbed into the appropriate platform and finally polymerized under the UV light. The two approaches can serve in vitro studies of multiple fields (e.g. pharmaceutic, biological studies, tissue engineering, biotechnology) or commercial purposes (e.g. cell expansion, growth factors production). In some instances, the present disclosure is used as a coating. For such purpose, the hydrogel may be applied in the liquid form and then polymerized under UV light. The described technique can be used to coat, for example, cell culture platforms, scaffolds or medical devices to improve their biocompatibility and performance. In some instances, the present disclosure is used as a delivery matrix. For such purpose, the hydrogel may be loaded via covalent bonds, non-covalent bonds or entrapment of cells or therapeutic molecules, which are then delivered into specific sites, like, for example, injured tissues. In particular the hydrogel is loaded with, but not limited to, stem cells, tissue-specific cells, peptides, proteins, vaccines, antibodies, growth factors, drugs and DNA.

In some instances, the present disclosure is used as an injectable system. Injectable system can be used alone or combined with cells or therapeutic molecules. For such purpose, the hydrogel may be injected into the patient at the site of injury or defect and polymerized in situ. Alternatively, the hydrogel may be injected as a bioink, or a bioink component, by a bioprinter, or similar apparatus, to produce constructs with controlled structures.

In some instances, the present disclosure is used as an implantable or adhesive construct. For such purpose, the hydrogel may be configured, for example, as microparticles, capsules, fibers, membranes, discs, patches, among others. In any of these configurations the hydrogel may be further combined with other materials, cells or therapeutic molecules.

In some instances, the present disclosure can be incorporated into microfluidic, microarray or lab-on-a-chip platforms.

The term “comprising” whenever used in this document is intended to indicate the presence of stated features, integers, steps, components, but not to preclude the presence or addition of one or more other features, integers, steps, components or groups thereof.

It will be appreciated by those of ordinary skill in the art that unless otherwise indicated herein, the particular sequence of steps described is illustrative only and can be varied without departing from the disclosure. Thus, unless otherwise stated the steps described are so unordered meaning that, when possible, the steps can be performed in any convenient or desirable order.

The disclosure should not be seen in any way restricted to the embodiments described and a person with ordinary skill in the art will foresee many possibilities to modifications thereof.

The above described embodiments are combinable.

The following claims further set out particular embodiments of the disclosure.

### REFERENCES

1. Huang, G., et al., Accelerated expansion of epidermal keratinocyte and improved dermal reconstruction achieved by engineered amniotic membrane. Cell Transplant, 2013. 22(10): p. 1831-44.
2. Hariya, T., et al., Transparent, resilient human amniotic membrane laminates for corneal transplantation. Biomaterials, 2016. 101: p. 76-85.
3. Adamowicz, J., et al., New Amniotic Membrane Based Biocomposite for Future Application in Reconstructive Urology. PLoS One, 2016. 11(1): p. e0146012.
4. Murphy, S. V., et al., Solubilized Amnion Membrane Hyaluronic Acid Hydrogel Accelerates Full-Thickness Wound Healing. Stem Cells Transl Med, 2017. 6(11): p. 2020-2032.
5. Ryzhuk, V., et al., Human amnion extracellular matrix derived bioactive hydrogel for cell delivery and tissue engineering. Materials Science and Engineering C, 2018. 85(December 2017): p. 191-202.

1. A hydrogel composition comprising:
a protein extract obtained from a decellularized human placental membrane with a polymerizable or assemblable moiety; and
at least a photoinitiator;
wherein the polymerizable or assemblable moiety is selected from the group consisting of: a methacrylate, acrylate, ethacrylate, acryloyl, thiol, acrylamide, aldehyde, azide, cyclic oligosaccharides, phenol, phenol derivatives, and combinations thereof; and
wherein the polymerizable or assemblable moiety is bound to the protein extract obtained from the decellularized human placental membrane.

2. The hydrogel composition of claim 1, wherein the ratio of protein extract:polymerizable or assemblable moiety is from 10:1×10⁻⁵ (v/v) to 10:1×10⁻¹.

3. The hydrogel composition of claim 1, wherein the concentration of protein extract varies from 1-15% w/V.

4. The hydrogel composition of claim 1, wherein the protein extract comprises at least two of the following proteins: keratin, collagen, desmoplakin, dermcidin and peroxiredoxin.

5. (canceled)

6. (canceled)

7. The hydrogel composition of claim 1, wherein the polymerizable or assemblable moiety is methacrylate.

8. The hydrogel composition of claim 1, wherein the polymerizable or assemblable moiety is a thiol, a methacrylate, or mixtures thereof.

9. The hydrogel composition of claim 1, wherein the polymerizable or assemblable moiety is a phenol or phenol derivative.

10. The hydrogel composition of claim 1, wherein the decellularized human placental membrane is an amnion membrane, chorion membrane or combinations thereof.

11. (canceled)

12. The hydrogel composition of claim 1, wherein the polymerization occurs by crosslinking performed via chemical crosslinking, non-covalent bonds, or crosslinked enzymatically via transglutaminase, or combinations thereof.

13. The hydrogel composition of claim 1, wherein the photoinitiator is selected from the group consisting of: 2-Hydroxy-4′-(2-hydroxyethoxy)-2-methylpropiophenone, acetophenone, benzil, benzophenone, and 1-hydroxycyclohexyl phenyl ketone.

14. The hydrogel composition of claim 1, wherein the protein extract is chemically modified with biodegradable linkages.

15. The hydrogel composition of claim 1, further comprising inorganic materials selected from the group consisting of: calcium phosphate, magnetic particles, metallic nanoparticles, bioglass particles, fibers and combinations thereof.

16. The hydrogel composition of claim 1, further comprising chitosan, alginate, laminarin, hyaluronic acid, or polyethylene glycol, or combinations thereof.

17. The hydrogel composition of claim 1, wherein the protein extract obtained from a decellularized human placental membrane is an autologous protein.

18. (canceled)

19. (canceled)

20. (canceled)

21. (canceled)

22. (canceled)

23. (canceled)

24. (canceled)

25. (canceled)

26. (canceled)

27. (canceled)

28. A method for obtaining a protein extract of decellularized placental membrane comprising the following steps:
washing an isolated placental membrane;
decellularizing the washed membrane;
solubilizing the decellularized membrane;
freezing, lyophilizing and/or grinding the solubilized membrane; and
bounding to the protein extract at least one polymerizable or assemblable moiety to obtain a reactively enhanced extract.

29. The method of claim 28, further comprising the step of storing the functionalized protein extract at between around −80° C. and 4° C.

30. The method of claim 28, further comprising the following steps:
conjugation with a photoinitiator selected from the group consisting of: 2-Hydroxy-4′-(2-hydroxyethoxy)-2-methylpropiophenone, acetophenone, benzil, benzophenone, and 1-hydroxycyclohexyl phenyl ketone; and
chemically modifying the protein extract derived from the placental membrane with biodegradable linkages.

31. The method of claim 28, further comprising the step of terminally sterilizing the obtained protein extract derived from the placental membrane by means of filtration or chemical reaction.

32. The method of claim 28, wherein the decellularization step is performed by means of detergent, enzymatical methods, chemical methods, physical methods, or combinations thereof.

33. The method of claim 28, wherein the solubilization step is performed by enzymatic digestion.

34. (canceled)
